(19) United States
(12) Patent Application Publication
(10) Pub. No.: US 2022/0233415 A1
(43) Pub. Date: Jul. 28, 2022

(54) Solid Oral Care Compositions

(71) Applicant: Colgate-Palmolive Company, New York, NY (US)

(72) Inventors: Najma KHAN, Somerset, NJ (US); Jamal BERRY, Jackson, NJ (US); Shashank Potnis, North Brunswick, NJ (US)

(73) Assignee: Colgate-Palmolive Company, New York, NY (US)

(21) Appl. No.: 17/583,562

(22) Filed: Jan. 25, 2022

Related U.S. Application Data
(60) Provisional application No. 63/141,255, filed on Jan. 25, 2021.

(51) Int. Cl. A61K 8/21; A61K 8/02; A61K 8/73; A61K 8/81; A61K 8/34; A61K 8/24; A61K 8/23; A61K 8/25; A61K 8/19; A61Q 11/00

(52) U.S. Cl. CPC A61K 8/21; A61K 8/0216; A61K 8/731; A61K 8/8176; A61K 8/345; A61K 2800/28; A61K 8/23; A61K 8/25; A61K 8/19; A61Q 11/00; A61K 8/24

(57) ABSTRACT

This invention relates to solid oral care compositions, for example a tablet, comprising a binder system. In certain aspects the invention is directed to solid oral care compositions that are tablets that comprise a binder system, wherein the binder system comprises polyvinyl pyrrolidone and hydroxyethyl cellulose, and to methods of using and of making these compositions.

### FIELD OF THE INVENTION

This invention relates to solid oral care compositions, for example tablet comprising a binder system. In certain aspects the invention is directed to solid oral care compositions that are tablets that comprise a binder system, wherein the binder system comprises polyvinylpyrrolidone and hydroxyethyl cellulose, and to methods of using and of making these compositions.

### BACKGROUND

An acceptable dentifrice composition should remove debris, effectively clean the oral cavity, and effectively deliver any possible active ingredients that are included in the composition. This mode of action serves to aid in the prevention of tooth decay and promoting gingival health. Typically, it is necessary to brush with a dentifrice that contains various cleaning agents, actives and abrasives. The paste form is a common and popular form as a tooth cleaning product and generally is provided by filling laminate tubes with the paste, and the rheological properties can make these types of products desirable for purposes of delivery active ingredients. There can be issues with dispensing this type of product and efficiently using all of the product that is provided in the tube. For instance, the viscosity of the paste can make it may be difficult to squeeze the last drop of the toothpaste from the tube sufficiently. Shipping may also cause the tubes to deform which, in turn, can affect dispensing the product.

A number of dentifrices on the market are in paste or gel format and packaged in a tube, squeeze bottle, pressurized can, or pump dispenser. While these dentifrices have been traditionally used, there is a market need for products that can be used on the go, require less packaging, can be stored long term and require less water. However, the types of oral care products which address these needs appears to be relatively limited. Tablets are available, but some of these products may have issues with characteristics such as friability, and may break or fall apart during shipment or at some time prior to use by the consumer. Other issues with these products may be the ability to deliver various active ingredients to a consumer's teeth or gums at the same level as toothpastes and gels.

Accordingly, there is a need for an oral care product that can possibly be an alternative to the pastes and gels that are currently on the market.

### BRIEF SUMMARY

The invention is directed to a solid composition, e.g., a tablet, comprising a binder system, wherein the binder system comprises polyvinylpyrrolidone (PVP) and hydroxyethyl cellulose (HEC). In one aspect, the PVP and HEC are in a weight ratio of 0.5:1 to 2.5:1 (PVP: HEC) (e.g., PVP: HEC in a weight ratio of 1.5:1). In yet a further aspect, the solid composition (e.g., a single-use tablet) is able to deliver one or more active ingredients (e.g., fluoride) at least at parity relative to a similarly situated toothpaste or gel with similar amounts/concentrations of the active. Without being bound by theory, the particular ratio of PVP and HEC is believed to be important for suitable friability and active delivery characteristics, and the components of the solid oral care composition are present in an amount that the active (e.g.,) is able to be released more effectively. The present disclosure, in certain embodiments, concerns a storage-stable solid composition comprising a binder system, wherein the binder system comprises polyvinylpyrrolidone (PVP) and hydroxyethyl cellulose (HEC) and an active ingredient, wherein the ingredient remains stable in the solid composition.

Further areas of applicability of the present disclosure will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight relative to the total composition. The amounts given are based on the active weight of the material.

Open terms such as “include,” “including,” “contain,” “containing” and the like mean “comprising.” In this description, unless otherwise stated, the use of the singular also includes the plural.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Solid oral care compositions include, for example, powder (e.g., a free-flowing granulation), tablet, caplet (type of tablet), granule, pellet, wafer, film and bead.

As used herein, “effective amount” refers to an amount of a compound or composition sufficient to induce a positive benefit, a functional benefit to the oral care composition (e.g., providing suitable friability and active delivery characteristics) and/or an oral health benefit (e.g., acceptable delivery of fluoride).

As used here, “unit-dose” refers to an amount of the oral care composition to be administered to a patient or consumer in a single use. The unit-dose oral care composition can be a unit-dose powder (e.g., a free-flowing granulation), unit-dose tablet, unit-dose caplet (type of tablet), unit-dose granule, unit-dose pellet, unit-dose wafer, unit-dose film and unit-dose bead or any other suitable unit-dose oral care composition capable of being retained in the oral cavity for a time sufficient to contact some or all of the dental surfaces and/or oral tissues for purposes of oral health.

In certain aspects, the solid oral care compositions of the disclosure may be stored in an air tight, moisture-proof package, e.g., sachets, sealed metal foil pouches, blister packs, and desiccant capped tubes. Useful packaging materials include polymeric packaging (e.g., polyethylene and polypropylene), metal foil packaging (e.g., aluminum), and combinations thereof.

The solid oral care compositions of the present disclosure contain no water or have a low water content. As used herein, the term “low water content” means the total concentration of water, including any free water and all water contained in any ingredients. In various embodiments of the composition, the amount of water is in an amount of less than 4% by weight, or less than 3% by weight, or less than 2% by weight, or less than 1% by weight, or less than 0.5% by weight, or less than 0.1%, or about 0.0001% to about 4% by weight, or about 0.0001% to about 0.5% by weight or about 0.0001% to about 0.1% by weight.

The solid oral care compositions of the disclosure (e.g., any of Composition 1, et seq) of the present disclosure can be in a variety of forms including, e.g., powder (e.g., a free-flowing granulation), tablet, caplet (type of tablet), granule, pellet, wafer, film and bead.

In certain aspects, the solid oral care compositions of the disclosure (e.g., any of Composition 1, et seq) of the present disclosure include one or more drying agents, for example, a hygroscopic material. Examples of drying agents include, but are not limited to, phosphates, pyrophosphates and other polyphosphates, calcium lactate, calcium lactophosphate, double salts of calcium lactate and mixtures thereof. Other drying agents include silica gels and precipitates (e.g., non-abrasive silicas); aluminas; and mixtures thereof. Specific examples include, but are not limited to, dicalcium phosphate dihydrate, calcium pyrophosphate, tricalcium phosphate, calcium polymetaphosphate, insoluble sodium polymetaphosphate, potassium metaphosphate, tricalcium phosphate, trimagnesium phosphate, and magnesium orthophosphate, hydrated alumina, aluminum silicate, zirconium silicates, bentonite, beta calcium pyrophosphate, or calcium carbonate. Pyrophosphate salts may also be used in the present invention as anticalculus agents or as buffering agents. Pyrophosphate salts suitable for the present compositions include dialkali metal pyrophosphate salts, tetra alkali metal pyrophosphate salts, and mixtures thereof. Disodium dihydrogen pyrophosphate, tetrasodium pyrophosphate, and tetrapotassium pyrophosphate in their unhydrated as well as hydrated forms are the preferred species. In various embodiments, the drying agents are about 0.1% to about 60%, about 1% to about 30%, about 1% to about 10%, or about 1% to about 5% by weight of the total composition, or about 2%, about 3%, about 4% or about 5%.

In certain aspects, the solid oral care compositions of the disclosure (e.g., any of Composition 1, et seq) of the present disclosure contain a buffering agent. Examples of buffering agents include anhydrous carbonates such as sodium carbonate, sesquicarbonates, bicarbonates such as sodium bicarbonate, silicates, bisulfates, phosphates such as monopotassium phosphate and dipotassium phosphate, citrates, pyrophosphates (sodium and potassium salts) and combinations thereof.

In certain aspects, solid oral care compositions of the disclosure (e.g., any of Composition 1, et seq) of the invention further comprise a disintegrating agent. Disintegrating agents include natural starches, such as maize starch, potato starch etc., directly compressible starches such as starch 1500, modified starches such as carboxymethyl starches and sodium starch glycolate which are available as PRIMOJEL® and EXPLOTAB® and EXPLOSOL® and starch derivatives such as amylose. Other examples are cross-linked polyvinylpyrrolidones, e.g. crospovidones available as e.g. POLYPLASDONE XL® and KOLLIDON XL®; modified celluloses such as cross-linked sodium carboxymethylcelluloses available as, e.g., AC-DI-SOL®, PRIMELLOSE®, PHARMACEL XL®, EXPLOCEL®, and NYMCEL ZSX®; alginic acid and sodium alginate; microcrystalline cellulose, e.g. AVICEL®, PHARMACEL®, EMCOCELL®, VIVAPUR®; and methacrylic acid-divinylbenzene copolymer salts available as e.g., AMBERLITE® IRP-88. Other examples of the disintegrating agent are light silicic anhydride, calcium silicate, magnesium metasilicate aluminate, and carboxymethyl cellulose. In the present invention, each of them may be used solely or two or more thereof may be used jointly. Typical amounts of disintegrating agent are about 0.5% to about 20%, in one embodiment about 1% to about 5%, in another embodiment about 1% to about 3%, by weight of the total composition.

In certain aspects, the binder system of the solid oral care compositions of the disclosure (e.g., any of Composition 1, et seq) further comprises a polymeric binder which adds bulk to the compositions and assists in holding the components of the composition together when in the form of a tablet. Examples of suitable polymeric binders include, e.g., starches, natural gums, (e.g., xanthan gum), cellulose gums, microcrystalline cellulose, maltodextrins, methylcellulose, cellulose ethers, sodium carboxymethylcellulose, ethylcellulose, gelatin, polyethylene glycol, pectins, alginates, polyacrylamides, polyvinyloxozolidone, polyvinyl alcohols and mixtures thereof. In certain aspects, the binder system can also comprise one or more non-polymeric binders such as dextrose, lactose, sucrose, sorbitol, mannitol, and xylitol.

In certain aspects, the solid oral care compositions of the disclosure (e.g., any of Composition 1, et seq) of the invention can include an acid buffering agent. For example, these acids can include citric acid, ascorbic acid, malic acid, adipic acid, tartaric acid, fumaric, succinic acid, sodium acid pyrophosphate, lactic acid, hexamic acid, and acid salts and acid anhydrides thereof, and mixtures thereof. Examples of useful acid anhydrides include citraconic anhydride, glucono-D-lactone, and succinic anhydride. Examples of useful acid salts include potassium bitartrate, acid citrate salts, sodium dihydrogen phosphate, disodium dihydrogen phosphate, sodium acid sulfite, and combinations thereof.

In certain aspects, the solid oral care compositions of the disclosure (e.g., any of Composition 1, et seq) of the invention can include a carbonate base. Examples of suitable carbonate bases include sodium bicarbonate, sodium carbonate, sodium sesquicarbonate, potassium carbonate, potassium bicarbonate, calcium carbonate, magnesium carbonate, magnesium oxide, sodium glycine carbonate, L-lysine carbonate, arginine carbonate, zinc carbonate, zinc oxide and mixtures thereof. In certain aspects, the base is present in the composition in an amount of 5% by weight to 60% by weight, about 7% by weight to 50% by weight, or about 8% by weight to about 15% by weight.

In certain aspects, the solid oral care compositions of the disclosure (e.g., any of Composition 1, et seq) of the invention comprise a lubricant. Various lubricants are suitable for use in the composition including water dispersible, water soluble, water insoluble lubricants and combinations thereof. Examples of useful water soluble lubricants include sodium benzoate, polyethylene glycol, L-leucine, adipic acid, and combinations thereof. The composition can also include water insoluble lubricants including, e.g., stearates (e.g., magnesium stearate, calcium stearate and zinc stearate), oils (e.g., mineral oil, hydrogenated and partially hydrogenated vegetable oils, and cotton seed oil) and combinations thereof. Other water insoluble lubricants include, e.g., animal fats, polyoxyethylene monostearate, talc, and combinations thereof. When the composition is in the form of a tablet, the composition preferably includes a sufficient amount of lubricant to enable the composition to be formed into tablets and released from a high speed tableting press in the form of a tablet. In certain aspects, the amount of lubricant in the composition is from 1% by weight to about 15% by weight, from 1% by weight to about 12% by weight, from 2% by weight to about 10% by weight, or from 3% by weight to about 8% by weight (e.g., about 4%).

In certain aspects, the solid oral care compositions of the disclosure (e.g., any of Composition 1, et seq) described herein can further include additional ingredients, e.g., flavor agents; fillers; surfactants; preservatives, e.g., sodium benzoate and potassium sorbate; and dyes and pigments; and sweeteners.

In certain aspects, the solid oral care compositions of the disclosure (e.g., any of Composition 1, et seq) may comprise anionic surfactants, e.g., the Compositions of Composition 1, et seq., for example, water-soluble salts of higher fatty acid monoglyceride monosulfates, such as the sodium salt of the monosulfated monoglyceride of hydrogenated coconut oil fatty acids such as sodium cocoyl glutamate, sodium N-methyl N-cocoyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOS0₃X, wherein m is 6-16, e.g., 10, n is 1-6, e.g., 2, 3 or 4, and X is Na or, for example sodium laureth-2 sulfate (CH₃(CH₂)₁₀CH₂(OCH₂CH₂)₂OS0₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C_{6-3O} alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., 1.5%.

In another aspect, solid oral care compositions of the disclosure (e.g., any of Composition 1, et seq) can comprise one or more cationic surfactants. Cationic surfactants that are useful in the present invention can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants of the disclosure, e.g., any of Composition 1 et seq., that can be used in the compositions of the disclosure can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the invention comprises a nonionic surfactant selected from poloxamers (e.g., poloxamer 407), polysorbates (e.g., polysorbate 20), polyoxyl hydrogenated castor oils (e.g., polyoxyl 40 hydrogenated castor oil), and mixtures thereof.

The surfactant or mixtures of compatible surfactants can be present in the compositions of the present invention in 0.1% to 5%, in another embodiment 0.3% to 3% and in another embodiment 0.5% to 2.5% by weight of the total composition.

In certain aspects, the solid oral care compositions of the disclosure (e.g., any of Composition 1, et seq) described herein can comprise one or more fillers. For example, the filler can be one or more selected from: crystalline cellulose, ethylcellulose, dextrin, various kinds of cyclodextrin (α-cyclodextrin, β-cyclodextrin and γ-cyclodextrin), sodium sulfate, as well as derivatives thereof and pullulan.

In certain aspects, solid oral care compositions of the disclosure (e.g., any of Composition 1, et seq) can further comprise one or more flavoring agents. Useful flavor agents include natural and synthetic flavoring sources including, e.g., volatile oils, synthetic flavor oils, flavoring aromatics, oils, liquids, oleoresins and extracts derived from plants, leaves, flowers, fruits, stems and combinations thereof. Suitable flavor agents include, e.g., citric oils, e.g., lemon, orange, grape, lime and grapefruit, fruit essences including, e.g., apple, pear, peach, grape, strawberry, raspberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof.

In certain aspects, the solid oral care compositions of the disclosure (e.g., any of Composition 1, et seq) described herein can comprises one or more dyes, lakes. Useful lakes include dyes absorbed on aluminum hydroxide and other suitable carriers.

In certain aspects, solid oral care compositions of the disclosure (e.g., any of Composition 1, et seq) described herein can comprise one or more sweetener, e.g., selected from: stevia, sugars such as sucrose, glucose, invert sugar, fructose, ribose, tagalose, sucralose, malitol, erythritol, xylitol, and mixtures thereof, saccharin and its various salts (e.g., sodium and calcium salt of saccharin), cyclamic acid and its various salts, dipeptide sweeteners (e.g., aspartame), acesulfame potassium, dihydrochalcone, glycyrrhizin, and sugar alcohols including, e.g., sorbitol, sorbitol syrup, mannitol and xylitol, and combinations thereof.

In certain aspects, the solid oral care compositions of the disclosure (e.g., any of Composition 1, et seq) may comprise a calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄•2H₂O, also sometimes referred to herein as DiCal) or calcium pyrophosphate. Alternatively, calcium carbonate, and in particular natural calcium carbonate or precipitated calcium carbonate, may be employed as an abrasive.

In certain aspects, the solid oral care compositions of the disclosure (e.g., any of Composition 1, et seq) may include one or more additional particulate materials, for example silica abrasives such as precipitated silicas having a mean particle size of up to about 20 microns, such as Zeodent 115®, marketed by J. M. Huber. Other useful abrasives also include sodium metaphosphate, potassium metaphosphate, aluminum silicate, calcined alumina, bentonite or other siliceous materials, or combinations thereof.

The silica abrasive polishing materials useful herein, as well as the other abrasives, generally have an average particle size ranging between about 0.1 and about 30 microns, about between 5 and about 15 microns. The silica abrasives can be from precipitated silica or silica gels, such as the silica xerogels described in U.S. Pat. No. 3,538,230, to Pader et al. and U.S. Pat. No. 3,862,307, to Digiulio, both incorporated herein by reference. Particular silica xerogels are marketed under the trade name Syloid® by the W. R. Grace & Co., Davison Chemical Division. The precipitated silica materials include those marketed by the J. M. Huber Corp. under the trade name Zeodent®, including the silica carrying the designation Zeodent 115 and 119. These silica abrasives are described in U.S. Pat. No. 4,340,583, to Wason, incorporated herein by reference.

In certain aspects, the solid oral care compositions of the disclosure (e.g., any of Composition 1, et seq) comprises an abrasive. For example, the abrasive may include silica gels and precipitated amorphous silica having an oil absorption value of about less than 100 cc/100 g silica and in the range of about 45 cc/100 g to about 70 cc/100 g silica. Oil absorption values are measured using the ASTA Rub-Out Method D281. In certain embodiments, the silicas are colloidal particles having an average particle size of about 3 microns to about 12 microns, and about 5 to about 10 microns.

In certain aspects, the particulate or abrasive materials comprise a large fraction of very small particles, e.g., having a d50 less than about 5 microns, for example small particle silica (SPS) having a d50 of about 3 to about 4 microns, for example Sorbosil AC43® (Ineos). Such small particles are particularly useful in formulations targeted at reducing hypersensitivity. The small particle component may be present in combination with a second larger particle abrasive. In certain embodiments, for example, the formulation comprises about 3 to about 8% SPS and about 25 to about 45% of a conventional abrasive.

Low oil absorption silica abrasives particularly useful in the practice of the invention are marketed under the trade designation Sylodent XWA® by Davison Chemical Division of W.R. Grace & Co., Baltimore, Md. 21203. Sylodent 650 XWA®, a silica hydrogel composed of particles of colloidal silica having a water content of about 29% by weight averaging about 7 to about 10 microns in diameter, and an oil absorption of less than about 70 cc/100 g of silica is an example of a low oil absorption silica abrasive useful in the practice of the present invention. The total abrasive content is present in the oral care composition of the present invention at a concentration of about 10 to about 60% by weight, in other embodiment about 15 to about 35% by weight, and in another embodiment about 15 to about 25% by weight.

Natural calcium carbonate is found in rocks such as chalk, limestone, marble and travertine. It is also the principle component of egg shells and the shells of mollusks. The natural calcium carbonate abrasive of the invention is typically a finely ground limestone which may optionally be refined or partially refined to remove impurities. For use in the present invention, the material has an average particle size of less than 10 microns, e.g., 3-7 microns, e.g. about 5.5 microns. Because natural calcium carbonate may contain a high proportion of relatively large particles of not carefully controlled, which may unacceptably increase the abrasivity, preferably no more than 0.01%, preferably no more than 0.004% by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D₉₀ of 3-6 microns, for example 3.8=4.9, e.g., about 4.3; a D₅₀ of 1-4 microns, e.g. 2.2-2.6 microns, e.g., about 2.4 microns, and a D₁₀ of 1-2 microns, e.g., 1.2-1.4, e.g. about 1.3 microns. The particles have relatively high-water absorption, e.g., at least 25 g/100 g, e.g. 30-70 g/100 g. Examples of commercially available products suitable for use in the present invention include, for example, Carbolag® 15 Plus from Lagos Industria Quimica.

In certain aspects, the solid oral care compositions of the disclosure (e.g., any of Composition 1, et seq) can comprise one or more polymers, such as polyethylene glycols, polyvinylmethyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include about 1:4 to about 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

Another useful class of polymeric agents includes polyamino acids, particularly those containing proportions of anionic surface-active amino acids such as aspartic acid, glutamic acid and phosphoserine, as disclosed in U.S. Pat. No. 4,866,161 Sikes et al., incorporated herein by reference.

In certain aspects, the solid oral care compositions of the disclosure (e.g., any of Composition 1, et seq) may further comprise one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., incorporated herein by reference.

Representative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof.

In certain embodiments, the solid oral care composition of the disclosure (e.g., Composition 1, et seq) may comprise a source of fluoride ions or fluorine-providing ingredient in amounts sufficient to supply about 25 ppm to 25,000 ppm of fluoride ions, generally at least about 500 ppm, e.g., about 500 to about 2000 ppm, e.g., about 1000 to about 1600 ppm, e.g., about 1450 ppm. The appropriate level of fluoride will depend on the particular application.

Fluoride ion sources may be added to the compositions of the invention at a level of about 0.01 wt. % to about 10 wt. % in one embodiment or about 0.03 wt. % to about 5 wt. %, and in another embodiment about 0.1 wt. % to about 1 wt. % by weight of the composition in another embodiment. Weights of fluoride salts to provide the appropriate level of fluoride ion will obviously vary based on the weight of the counter ion in the salt.

In preparing oral care compositions, it is sometimes necessary to add some thickening material to provide a desirable consistency or to stabilize or enhance the performance of the formulation. In certain aspects, the solid oral care compositions (e.g., any of Composition 1, et seq) can comprise one or more thickening agents selected from: carboxyvinyl polymers, carrageenan, and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 10.0% by weight of the total composition are used.

It is understood that while general attributes of each of the above categories of materials may differ, there may be some common attributes and any given material may serve multiple purposes within two or more of such categories of materials. All of the ingredients in the compositions may have functions in addition to their primary function, and may contribute to the overall properties of the composition, including its stability, efficacy, consistency, mouthfeel, taste, odor and so forth. For example, a binder may also function as a disintegrating agent and vice versa.

In certain aspects, the solid compositions of the present disclosure can be made via techniques known in the art. Documents which disclose techniques which may be used to prepare the solid compositions of the present disclosure are U.S. Pat. Nos. 4,886,669; 6,106,861; 6,596,311; 6,743,443; 6,811,793; 7,501,409; 7,815,897; 8,377,995; and US patent application 2005/0169986, all of which are incorporated herein by reference in their entireties. In general, the ingredients and optional components can be kneaded with an organic solvent, filled in a mold and subjected to a compression-molding. The organic solvent can be an alcohol such as methanol, ethanol, propanol, isopropanol. The kneading and granulating operations carried out by adding such auxiliary agents for making the preparation and by adding such a solvent may be conducted using the conventionally used apparatus. For example, a fluidized bed granulator, a tumbling granulator, an extrusion granulator or a spray-drying drier may be used. The solid compositions may also be prepared via freeze drying.

In certain aspects, granules can be prepared by any one of known methods for preparing granules such as dry granulation, layering granulation, impregnated-granulation, etc. For dry granulation, a mixture of ingredients with optional additive(s) is subjected to granulation with a roller compactor, a roll granulator, etc.

For layering granulation, a mixture similar to the above is added to a rolling inactive carriers while spraying a binder solution with a centrifugal fluidized bed granulator or the like to make the mixture adhere to the carries. Examples of the inactive carrier that used in this method include crystals of sugars or inorganic salts such as crystalline lactose, crystalline cellulose, crystalline sodium chloride, etc., and spherical granules such as spherical granules of crystalline cellulose (brand name: Avicel SP, Asahi Kasei Corporation), spherical granules of crystalline cellulose and lactose (brand name: Nonpareil-NP-5 and NP-7, Freund Co., Ltd.), spherical granules of purified white sugar (brand name: Nonpareil-103, Freund Co., Ltd.), spherical granules of lactose and a starch, etc.

For impregnating granulation, a solution containing potassium peroxymonosulfate and other ingredients at an appropriate concentration is mixed with porous carriers thereby a sufficient amount of solution is made to retain in the cavities of the carrier, which is followed by drying to remove the solvent. Examples of the porous carrier that can be used include magnesium aluminometasilicate (brand name: Neusiline, Fuji Chemical Industry Co., Ltd.), calcium silicate (Florite, Eisai Co., Ltd.), etc. Examples of the solvent include ethanol, methanol, or the like.

In one aspect, the disclosure is directed to a solid oral care composition (Composition 1) (e.g., a tablet) comprising a binder system, wherein the binder system comprises polyvinylpyrrolidone (PVP) and hydroxyethyl cellulose (HEC).

For example, the invention contemplates any of the following compositions (unless otherwise indicated, values are given as percentage of the overall weight of the composition):
1.1. The solid oral care composition of Composition 1, wherein the binder system comprises PVP and HEC in a weight ratio of 0.5:1 to 2.5:1 (PVP: HEC) (e.g., 0.75:1 to 2:1) (e.g., 1:1 to 1.75:1) (e.g., 1.25:1 to 1.75:1) (e.g., 0.75:1 or 1:1 or 1.25:1 or 1.5:1 or 1.75:1 or 2:1), wherein the weight is relative to the total weight of the composition.
1.2. Composition 1 or 1.1, wherein the PVP and HEC are in a weight ratio of 1.5:1 (PVP: HEC), wherein the weight is relative to the total weight of the composition.
1.3. Any of the preceding compositions, wherein the amount of PVP is from 1%-15% by weight of the total composition.
1.4. Any of the preceding compositions, wherein the amount of PVP is from 3%-8% by wt. of the total composition (e.g., about 6%).
1.5. Any of the preceding compositions, wherein the amount of PVP is 6% by wt. of the total composition.
1.6. Any of the preceding compositions, wherein the amount of HEC is from 0.5%-10% by weight of the total composition.
1.7. Any of the preceding compositions, wherein the amount of HEC is from 1%-5% by wt. of the total composition (e.g., from 2%-5% by wt.) (e.g., about 4%).
1.8. Any of the preceding compositions, wherein the amount of HEC is 4% by wt. of the total composition.
1.9. Any of the preceding compositions comprising PVP in an amount of about 6% by wt. and HEC in an amount of about 4% by wt., relative to the total weight of the composition.
1.10. Any of the preceding compositions, wherein the PVP is a cross-linked polyvinylpyrrolidone (e.g., crospovidones).
1.11. Any of the preceding compositions, wherein the composition further comprises a drying agent, and wherein the drying agent is selected from the group consisting of calcium lactate, calcium lactophosphate, double salts of calcium lactate, phosphates, pyrophosphates, polyphosphates, orthophosphates, metaphosphates, silica, alumina, bicarbonates, polymetaphosphates, aluminum silicate, zirconium silicates, bentonite, and combinations thereof.
1.12. Any of the preceding compositions, wherein the drying agent is selected from a pyrophosphate, alumina, sodium bicarbonate, and combinations thereof.
1.13. Any of the preceding compositions, wherein the composition is in the form of a tablet, powder or granule.
1.14. Any of the preceding compositions, wherein the composition is a single unit-dose oral care composition.
1.15. Any of the preceding compositions, wherein the composition contains no water or water in an amount of less than 4%, or less than 3%, or less than 2%, or less than 1%, or less than 0.5%, or from 0.0001% to 4%, or 0.0001% to 0.5% or 0.0001% to 0.1%, or 0.001% to 4%, by weight.
1.16. Any of the preceding compositions, wherein the drying agent is present in an amount of 0.1% to 60% by wt., 1% to 30% by wt., 1% to 11% by wt. (e.g., about 10% by wt. sodium bicarbonate).
1.17. Any of the preceding compositions further comprising a disintegrating agent selected from the group consisting of: natural starches, (e.g., maize starch, potato starch), directly compressible starch, modified starches (e.g., carboxymethyl starches and sodium starch glycolate), starch derivatives (e.g., amylose), modified celluloses (e.g., cross-linked sodium carboxymethylcelluloses), alginic acid, sodium alginate, microcrystalline cellulose, methacrylic acid-divinylbenzene copolymer salts, light silicic anhydride, calcium silicate, magnesium metasilicate aluminate, carboxymethyl cellulose, and mixtures thereof.
1.18. Any of the preceding compositions, binder system further comprises a polymeric binder which adds bulk to the compositions and assists in holding the components of the composition together when in the form of a tablet.
1.19. Composition 1.18, wherein the polymer binder is selected from starches, natural gums, (e.g., xanthan gum), cellulose gums, microcrystalline cellulose, maltodextrins, methylcellulose, cellulose ethers, sodium carboxymethylcellulose, ethylcellulose, gelatin, polyethylene glycol, cross-linked polyvinylpyrrolidone, pectins, alginates, polyacrylamides, polyvinyloxazolidone, polyvinyl alcohols and mixtures thereof;
1.20. Any of the preceding compositions, wherein binder further comprises a non-polymeric binder.
1.21. The composition of 1.20, wherein the non-polymeric binder is xylitol.
1.22. Any of the preceding compositions, wherein the binder system is present in the composition in an amount of from 5% by weight to about 20% by weight, or from about 7% by weight to about 15% by weight (e.g., from 8%-12% by wt.) (e.g., from 9%-11% by wt.) (e.g., about 10% by wt.)
1.23. The Composition of 1.22, wherein the binder system is present in an amount of 10% by wt. relative to the total composition.
1.24. Any of the preceding compositions, further comprising a buffering agent selected from an anhydrous carbonate (e.g., sodium carbonate), a sesquicarbonate, a bicarbonate (e.g., sodium bicarbonate), a silicate, a bisulfate, a citrate, a phosphate (e.g., monopotassium phosphate), dipotassium phosphate, and combinations thereof.
1.25. The composition of 1.24, wherein the buffering agent is in an amount of from 5.0% to about 20%, or about 7% to about 15%, or about 10%, by weight of the total composition.
1.26. The composition of 1.25, wherein the buffering is sodium bicarbonate.
1.27. Any of the preceding compositions, comprising an acid buffering agent selected from: citric acid, ascorbic acid, malic acid, adipic acid, tartaric acid, fumaric acid, succinic acid, sodium acid pyrophosophate, lactic acid, hexamic acid, citraconic anhydride, glucono-D-lactone, succinic anhydride, potassium bitartrate, acid citrate salts, sodium dihydrogen phosphate, disodium dihydrogen phosphate, and sodium acid sulfite.
1.28. Any of the preceding compositions further comprising a lubricant (e.g., an insoluble lubricant) wherein the lubricant is selected from the group consisting of: stearates (e.g., magnesium stearate, calcium stearate and zinc stearate), oils (e.g., mineral oil, hydrogenated and partially hydrogenated vegetable oils, and cotton seed oil), animal fats, polyoxyethylene monostearate, talc, and combinations thereof.
1.29. The composition of 1.28, wherein the lubricant is magnesium stearate.
1.30. Any of the preceding compositions, additionally comprising one or more flavor agents, one or more fillers, one or more surfactants, one or more dyes or lakes, or any combination of two or more thereof.
1.31. Any of the preceding compositions wherein the solid oral care composition comprises an abrasive and/or particulate, wherein the abrasive and/or particulate is selected from a calcium phosphate, calcium sulfate, natural calcium carbonate, precipitated calcium carbonate, silica (e.g., synthetic silica), and combinations thereof (e.g., from 10%-30% by wt.) (e.g., 15%-25% by wt) (e.g. about 20% by wt).
1.32. The composition of any of the preceding claims, wherein the abrasive and/or particulate is selected from dicalcium phosphate dihydrate, precipitated calcium carbonate, synthetic silica, and combinations thereof (e.g., from 10%-30% by wt.) (e.g., 15%-25% by wt.) (e.g., about 20% by wt.).
1.33. The composition of any of the preceding claims wherein the solid oral care composition comprises calcium carbonate (e.g., precipitated calcium carbonate) from 2%-6% by wt. (e.g., about 4% by wt.) and dicalcium phosphate (e.g., dicalcium phosphate dihydrate) from 8%-12% by wt. (e.g., about 10% by wt.).
1.34. Any of the preceding compositions, wherein the solid oral care composition comprises an anionic surfactant (e.g., from 1%-5% by wt.) (e.g, about 2.5% by wt.).
1.35. Any of the preceding compositions, wherein solid oral care composition comprises a anionic surfactant selected from: sodium cocoyl glutamate, sodium lauryl sulfate, sorbitan fatty acid ester, polyoxyethylene (20) sorbitan monooleate (Polysorbate 80 or Tween 80), polyethylene glycol fatty acid ester, polyoxyethylene sorbitan fatty acid ester, polyoxyethylene alkyl ether, polyoxyethylene polyoxypropylene alkyl ether, polyoxyethylene polyoxypropylene block copolymer, polyoxyethylene alkyl phenyl ether, polyoxyethylene castor oil, polyoxyethylene hydrogenated castor oil, polyoxyethylene sorbitol fatty acid ester, polyoxyethylene glycerol fatty acid ester, and combinations thereof (e.g., from 1%-5% by wt.) (e.g, about 2.5% by wt.).
1.36. The preceding composition, wherein the surfactant is sodium cocoyl glutamate.
1.37. Any of the preceding solid oral care compositions, wherein the composition comprises a nonionic surfactant.
1.38. The preceding composition, wherein the solid oral care composition comprises a nonionic surfactant selected from poloxamers (e.g., poloxamer 407), polysorbates (e.g., polysorbate 20), polyoxyl hydrogenated castor oils (e.g., polyoxyl 40 hydrogenated castor oil), and mixtures thereof (e.g., from 1%-5% by wt.) (e.g, about 2% by wt.).
1.39. Any of the preceding compositions, wherein the solid oral care composition comprises a fluoride source.
1.40. The preceding composition, wherein the fluoride source is selected from stannous fluoride, sodium fluoride, potassium fluoride, sodium monofluorophosphate, sodium fluorosilicate, ammonium fluorosilicate, amine fluoride, ammonium fluoride, and combinations thereof.
1.41. The preceding composition, wherein the fluoride source is sodium monofluorophosphate.
1.42. Any of the preceding compositions, wherein the fluoride source is in an amount from 0.5%-2% (e.g., about 0.76%) by wt. of the total composition.
1.43. Any of the preceding compositions, wherein the solid oral care composition is a tablet that comprises:
   From 8%-12% (e.g., about 10% by wt.) of the binder system comprising polyvinylpyrrolidone (PVP) and hydroxyethyl cellulose (HEC), wherein the PVP is in an amount from 3%-8% (e.g., 6%) by wt., relative to the total composition, and the HEC is in an amount from 2%-6% (e.g., 4%) relative to the total composition;
   From 15%-25% of an abrasive source comprising: silica, calcium carbonate (e.g., precipitated calcium carbonate) and dicalcium phosphate; and
   From 0.5%-2% by wt., (e.g., about 0.76%) of sodium monofluorophosphate, wherein all weights are relative to the total weight of the composition.
1.44. Any of the preceding solid oral care compositions comprising a fluoride source, wherein the fluoride source is provided in an amount effective to supply from 25 ppm to 25,000 ppm of fluoride ions to the oral cavity (e.g., from 500 to about 2000 ppm) (e.g., 1000 to 1600 ppm) (e.g., 1000 ppm) (e.g., about 1450 ppm).
1.45. Any of the preceding compositions, wherein the binder system consists of polyvinylpyrrolidone (PVP) and hydroxyethyl cellulose (HEC) (e.g., wherein the PVP is in an amount from 3%-8% (e.g., 6%) by wt., relative to the total composition, and the HEC is in an amount from 2%-6% (e.g., 4%) relative to the total composition).
1.46. Any of the preceding compositions, wherein the solid oral care composition is selected from the group consisting of: a powder (e.g., a free-flowing granulation), tablet, granule, pellet, wafer, film and bead.
1.47. Any of the preceding compositions, wherein the solid oral care composition is a tablet or granule.
1.48. Any of the preceding composition, wherein the solid oral care composition is a tablet.

The invention thus further encompasses methods of using any of Composition 1, et seq., to (i) reduce or inhibit formation of dental caries, (ii) reduce, repair or inhibit early enamel lesions, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) inhibit microbial biofilm formation in the oral cavity, (ix) raise and/or maintain plaque pH at levels of at least pH 5.5 following sugar challenge, (x) reduce plaque accumulation, (xi) treat dry mouth, and/or (xii) clean the teeth and oral cavity, comprising applying a Composition of the Invention to the oral cavity, e.g., by applying a Composition of the Invention (e.g., any of Composition 1, et seq) to the oral cavity of a patient in need thereof.

### EXAMPLES

Exemplary embodiments of the present disclosure will be illustrated by reference to the following examples, which are included to exemplify, but not to limit the scope of the present invention.

### Example 1

The following is a representative tablet (e.g., solid oral care composition) of the present invention:

TABLE 1
Representative Formula A (Tablet)

| Material | Amount (% by wt.) |
|---|---|
| Silica | 6.2 |
| Xylitol | 26.0 |
| Dicalcium Phosphate Dihydrate | 10.3 |
| Anionic Surfactant | 2.5 |
| Precipitated Calcium Carbonate | 4.0 |
| Sodium Bicarbonate | 10.35 |
| Insoluble lubricant | 4.0 |
| Hydroxyethyl cellulose | 4.0 |
| Nonionic Surfactant | 2.0 |
| D-mannitol | 20.2 |
| Polyvinylpyrrolidone | 6.0 |
| Sodium monofluorophosphate | 0.76 |
| Flavor and Sweetener | 3.65 |
| Total | ~100 |

### Example 2

It is expected that in assays to be performed to measure fluoride delivery that the solid oral care composition tablet of Representative Formula A will likely deliver the same or similar concentration of fluoride in the assay compared to toothpaste formulations containing sodium monofluorophosphate and comprising similar concentrations of fluoride.

The invention has been described above with reference to illustrative Examples, but it is to be understood that the invention is not limited to the disclosed embodiments. Alterations and modifications that would occur to one of skill in the art upon reading the specification are also within the scope of the invention, which is defined in the appended claims.

1. A solid oral care composition comprising a binder system, wherein the binder system comprises polyvinylpyrrolidone (PVP) and hydroxyethyl cellulose (HEC).

2. The solid oral care composition of claim 1, wherein the binder system comprises PVP and HEC in a weight ratio of 0.5:1 to 2.5:1 (PVP: HEC), wherein the weight is relative to the total weight of the composition.

3. The solid oral care composition of claim 1, wherein the PVP and HEC are in a weight ratio of 1.5:1 (PVP: HEC), wherein the weight is relative to the total weight of the composition.

4. The solid oral care composition of claim 1, wherein the amount of PVP is from 1%-15% by weight of the total composition.

5. The solid oral care composition of claim 1, wherein the amount of PVP is from 3%-8% by wt. of the total composition.

6. The solid oral care composition of claim 1, wherein the amount of HEC is from 0.5%-10% by weight of the total composition.

7. The solid oral care composition of claim 1, wherein the composition comprises PVP in an amount of about 6% by wt. and HEC in an amount of about 4% by wt., relative to the total weight of the composition.

8. The solid oral care composition of claim 1, wherein the composition contains water in an amount of less than 4% by wt.

9. The solid oral care composition of claim 1, wherein binder further comprises a non-polymeric binder.

10. The composition of claim 8, wherein the non-polymeric binder is xylitol.

11. The solid oral care composition of claim 1, wherein the composition comprises an abrasive and/or particulate, and wherein the abrasive and/or particulate is selected from calcium phosphate, calcium sulfate, natural calcium carbonate, precipitated calcium carbonate, silica, and combinations thereof.

12. The solid oral care composition of claim 1, wherein the solid oral care composition comprises an anionic surfactant.

13. The solid oral care composition of claim 12, wherein solid oral care composition comprises a anionic surfactant selected from: sodium cocoyl glutamate, sodium lauryl sulfate, sorbitan fatty acid ester, polyoxyethylene (20) sorbitan monooleate (Polysorbate 80 or Tween 80), polyethylene glycol fatty acid ester, polyoxyethylene sorbitan fatty acid ester, polyoxyethylene alkyl ether, polyoxyethylene polyoxypropylene alkyl ether, polyoxyethylene polyoxypropylene block copolymer, polyoxyethylene alkyl phenyl ether, polyoxyethylene castor oil, polyoxyethylene hydrogenated castor oil, polyoxyethylene sorbitol fatty acid ester, polyoxyethylene glycerol fatty acid ester, and combinations thereof.

14. The solid oral care composition of claim 1, wherein the solid oral care composition comprises a nonionic surfactant selected from poloxamers, polysorbates, polyoxyl hydrogenated castor oils, and mixtures thereof.

15. The solid oral care composition of claim 1, wherein the solid oral care composition comprises a fluoride source.

16. The solid oral care composition of claim 15 wherein the fluoride source is selected from stannous fluoride, sodium fluoride, potassium fluoride, sodium monofluorophosphate, sodium fluorosilicate, ammonium fluorosilicate, amine fluoride, ammonium fluoride, and combinations thereof.

17. The solid oral care composition of claim 16, wherein the fluoride source is provided in an amount effective to supply from 25 ppm to 25,000 ppm of fluoride ions to the oral cavity.

18. (canceled)

19. The solid oral care composition of claim 1, wherein the solid oral care composition is a tablet.

20. The solid oral care composition of claim 1, wherein the solid oral care composition is a tablet that comprises:
a. From 8%-12% of the binder system comprising polyvinyl pyrrolidone (PVP) and hydroxyethylcellulose (HEC), wherein the PVP is in an amount from 3%-8% by wt., relative to the total composition, and the HEC is in an amount from 2%-6% relative to the total composition;
b. From 15%-25% of an abrasive source comprising: silica, calcium carbonate and dicalcium phosphate; and
c. From 0.5%-2% by wt., of sodium monofluorophosphate,
wherein all weights are relative to the total weight of the composition.

21. A method of treatment comprising the use any of the compositions of claim 1, to (i) reduce or inhibit formation of dental caries, (ii) reduce, repair or inhibit early enamel lesions, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) inhibit microbial biofilm formation in the oral cavity, (ix) raise and/or maintain plaque pH at levels of at least pH 5.5 following sugar challenge, (x) reduce plaque accumulation, (xi) treat dry mouth, and/or (xii) clean the teeth and oral cavity, comprising applying the composition to an oral cavity.
